(19) United States
(12) Patent Application Publication
(10) Pub. No.: US 2010/0203524 A1
(43) Pub. Date: Aug. 12, 2010

(54) POLYMERASES AND METHODS OF USE THEREOF

(75) Inventors: J. William Efcavitch, San Carlos, CA (US); Jayson L. Bowers, Cambridge, MA (US); Philip R. Buzby, Brockton, MA (US); John F. Thompson, Warwick, RI (US)

Correspondence Address: BROWN RUDNICK LLP, ONE FINANCIAL CENTER, BOSTON, MA, 02111, US

(73) Assignee: Helicos Biosciences Corporation, Cambridge, MA (US)

(21) Appl. No.: 12/609,543

(22) Filed: Oct. 30, 2009

Related U.S. Application Data
(60) Provisional application No. 61/110,139, filed on Oct. 31, 2008.
(60) Provisional application No. 61/246,790, filed on Sep. 29, 2009.

(51) Int. Cl. C12Q 1/68; C12N 9/10

(52) U.S. Cl. 435/6; 435/193

(57) ABSTRACT

The invention generally relates to polymerases for efficient and controlled sequencing-by-synthesis reactions. In certain embodiments, the invention provides a polymerase enzyme including at least one mutation that enhances ability of the polymerase as compared to a wild-type polymerase to incorporate a nucleotide into a nascent strand of DNA or cDNA including at least one modified nucleotide.

### RELATED APPLICATIONS

The present invention claims the benefit of and priority to U.S. provisional patent application Ser. Nos. 61/110,139, filed Oct. 31, 2008, and 61/246,790, filed Sep. 29, 2009, the contents of each of which are incorporated by reference herein in their entirety.

### FIELD OF THE INVENTION

The invention generally relates to polymerases for efficient and controlled sequencing-by-synthesis reactions.

### BACKGROUND

Sequencing-by-synthesis involves template-dependent addition of nucleotides to a template/primer duplex. Nucleotide addition is mediated by a polymerase enzyme and added nucleotides may be labeled in order to facilitate their detection. Single molecule sequencing has been used to obtain high-throughput sequence information on individual DNA or RNA. See, Braslaysky, Proc. Natl. Acad. Sci. USA 100: 3960-64 (2003). Recently, all four Watson-Crick nucleotides may be added simultaneously, each with a different detectable label or nucleotides may be added one at a time in a step-and-repeat manner for imaging incorporations.

One issue that presents itself in sequencing-by-synthesis is the difficulty of determining the number of nucleotides in homopolymer stretches in the template sequence. One solution to that problem has been to provide nucleotides that feature a reversible inhibition of base extension. For example, a 3′ blocker may be incorporated into the added nucleotide or a cleavable inhibitor may be attached to the base portion of the nucleotide. Once those nucleotides are incorporated, no further base addition is possible until the inhibition is released, at which point the next incorporation can occur. While that approach solves the problem encountered with a highly-processive polymerase, it results in a nucleotide that is “scarred” upon release of inhibition. These “scarred” nucleotides often serve as poor substrates for the polymerase enzyme, making subsequent incorporations difficult or impossible. Thus, there is a tension between the desire to achieve step-and-repeat nucleotide incorporation and the desire for a highly-processive polymerase that will easily incorporate nucleotide analogs.

### SUMMARY

The present invention generally relates to altered polymerases that recognize and are capable of incorporating nucleotide analogs in a polymerase-mediated, template-based sequencing-by-synthesis reaction. In particular, modified polymerases of the invention recognize non-native nucleotides that contain an overhang or scar due to cleavage of a label or other inhibitory group upon incorporation and imaging. Accordingly, sequencing reactions are conducted with altered nucleotides designed to limit polymerase-mediated processivity. As a result, one is able to conduct a sequencing-by-synthesis reaction that allows incorporation of a single nucleotide for imaging prior to incorporation of subsequent nucleotides into the primer.

The invention provides polymerases that are altered to catalyze addition of nucleotide analogs into a primer/template duplex at rates that are similar or the same as those of incorporation of a native nucleotide by a wild-type polymerase. Nucleotide analogs, as described below, contain an alteration such that, once incorporated, they inhibit further nucleotide incorporation until the inhibition is released. Although these nucleotides typically remain altered (i.e., non-natural) after release of the inhibitory element, modified polymerases of the invention nonetheless recognize them as substrates for subsequent base incorporation upon removal of the inhibition. Mutated polymerases, while directed to accept nucleotide analogs, also will incorporate native nucleotides at acceptable rates. Nucleotide substrates for modified polymerases of the invention may be modified in numerous ways. For example, and without limitation, they may have modified 3′ hydroxyl groups, they may have modified sugar portions, or preferably they may have modifications in the base portion of the nucleotide, such as an “overhang” of between 2 and 15 atoms that result from cleavage of a label or inhibitor upon prior incorporation.

In certain embodiments, the modified nucleotide includes a detectable label connected to a nitrogenous base portion of a nucleotide by a cleavable linker. Exemplary detectable labels include radiolabels, florescent labels, enzymatic labels, etc. In particular embodiments, the detectable label may be an optically detectable label, such as a fluorescent label. Exemplary fluorescent labels include cyanine, rhodamine, fluorescien, coumarin, BODIPY, alexa, or conjugated multi-dyes.

An exemplary polymerase of the invention is a Stoffel fragment of Taq polymerase that is modified to incorporate subsequent nucleotides in a sequencing-by-synthesis reaction even if the upstream nucleotide substrate is a modified nucleotide. Another exemplary polymerase of the invention is a Taq polymerase that is mutated to incorporate a modified nucleotide into a strand containing a non-native or altered nucleotide in the template. Modified polymerases of the invention have enhanced ability to incorporate into a non-native template as compared to the wild-type.

In certain embodiments, the polymerase is a mutated Stoffel fragment of the Taq polymerase. For example, the mutated Stoffel fragment can include mutations at H784Q and T664A. In other embodiments, the mutated Stoffel fragment contains one or more mutations selected from F598I, I614F, V618I, L619M, I638V, T640A, A643G, M646V, A661T, T664V, I665V, L670M, A691V, F700Y, I753V, T756S, A757G, H784Q, V618I, L619M, V631A, I638V, T640K, M646V, M658L, A661I, T664A, I665V, L670M, F700Y, A757G, H784Q, I614F, L619M, L622F, T640E, M678K, T684A, M751T, V753I, T756S, A757G, and L760I.

Exemplary modified Taq polymerases contain one or more of the following mutations: mutations: V618I, L619M, V631A, I638V, T640K, M646V, M658L, A661I, T664A, I665V, L670M, F700Y, A757G, H784Q, I614F, L619M, L622F, T640E, M678K, T684A, M751T, V753I, T756S, A757G, L760I, V618I, L619M, V631A, I638V, T640K, M646V, M658L, A661I, T664A, I665V, L670M, F700Y, A757G, and H784Q.

Another embodiment of the invention provides a method of sequencing a nucleic acid, including directly or indirectly anchoring a nucleic acid duplex to a surface, the duplex including a template portion having a modified nucleotide at its 3′ terminus and a primer portion hybridized thereto; exposing the duplex to at least one detectably labeled nucleotide in the presence of a mutated polymerase capable of recognizing the nucleotide and the strand for incorporation as substrates in which the polymerase catalyzing addition of the labeled nucleotide to the primer portion in a template-dependent manner, detecting incorporation of the nucleotide into the primer portion, and repeating the exposing and detecting steps at least once. The template may be individually optically resolvable. The template or the primer may be attached to the surface. In a particular embodiment, the template is attached to the surface. In other embodiments, the polymerase may be attached to the surface.

The method may further include determining a sequence of the template based upon the order of incorporation of the labeled nucleotides. The method may further include removing unincorporated nucleotide and polymerase in all or some repetitions of the exposing and detecting steps. The method may further include neutralizing the label on the labeled nucleotide after the detecting step.

Another issue that presents itself in sequencing-by-synthesis is that synthetic or modified nucleotides and analogs, such as fluorescent dye labeled nucleotides, tend to be incorporated into a primer less efficiently than naturally-occurring dNTPs, due to the exonuclease activity of the polymerase. The reduced efficiency with which the non-native nucleotides are incorporated by the polymerase adversely affects the performance of sequencing techniques that depend upon faithful incorporation of such unconventional nucleotides, ultimately leading to sequencing termination. Methods of the invention solve the problem of premature terminations in nucleic acid synthesis reactions by providing polymerases that lack exonuclease activity or have reduced binding affinity of the 3′-5′ exonuclease domain.

Another aspect of the invention provides a method for increasing the accuracy of sequencing a nucleic acid including contacting a nucleic acid duplex having a primer nucleic acid hybridized to a template nucleic acid with a polymerase enzyme in the presence of a first labeled nucleotide under conditions that permit the polymerase to add nucleotides to said primer in a template-dependent manner, in which the polymerase lacks exonuclease activity or has a reduced binding affinity of the 3′-5′ exonuclease domain, detecting a signal from the incorporated labeled nucleotide, and sequentially repeating said contacting and detecting steps at least once, wherein sequential detection of incorporated labeled nucleotide determines the sequence of the nucleic acid.

The duplexes may be attached to a surface. The surface may include a plurality of duplex immobilized at different positions on the substrate. In certain embodiments, at least some of said duplex are individually optically resolvable.

In certain embodiments the polymerase is Klenow fragment of E. coli Pol 1 or Stoffel fragment of Taq polymerase. In other embodiments, the polymerase includes at least one mutation that results in reduced or eliminated exonuclease activity or reduced or eliminated binding affinity of the 3′-5′ exonuclease domain. For example, a polymerase of the invention includes at least one mutation that results in reduction of or absence of the 5′-3′ exonuclease activity or the 3′-5′ exonuclease activity.

In certain embodiments, the polymerase comprises at least one mutation that results in reduced or eliminated nucleic acid binding affinity of the 3′-5′ exonuclease domain of the polymerase. An example of such a polymerase is a klenow fragment including the following mutations: Leu361, Glu419, Lys422, Tyr423, Arg455, Phe473, and His660. In certain embodiments, one or more of those amino acids is substituted with an Ala.

An altered polymerase of the invention also contains mutations resulting in reduction in or elimination of 5′-3′, 3′-5′, and/or reduction in or elimination of 3′-5′ domain binding.

In other embodiments, the polymerase includes at least one mutation that affects metal ion binding. An example of a such a polymerase is a Klenow fragment including the following mutations: Asp355, Glu357, Asp424, and Asp501.

### BRIEF DESCRIPTION OF THE DRAWINGS

FIG. 1 is a set of schematics showing single molecule sequencing by synthesis substrates and a selection system. Panel A shows a modified dUTP (e.g., a fluorescently labeled uracil) used in sequencing (R═H) or selection experiments (R═CH₂NHCO-biotin). Panel B shows a representative scarred nucleotide, i.e., a nucleotide having a portion of a linker remaining attached to the nucleotide after cleavage of the detectable label. R₁═H at primer terminus and DNA when in remainder of primer. Panel C is a schematic showing an activity based phage display selection system. Active polymerase mutants incorporate a biotinylated nucleotide substrate into their intramolecularly attached oligonucleotide, allowing for recovery using streptavidin beads.

FIG. 2 is a set of schematics showing structure of Taq polymerase showing residues mutated in Taq197. The N- and O-helices are indicated with N and O, respectively. Panel A shows Thr664 is located 6.1 Å from the major groove of the incoming dNTP and His784 forms a hydrogen-bond with the ribosyl oxygen of the primer terminus. Panel B shows that three distinct networks of mutated residues (molecular surfaces shown) alter the packing between the N-helix and the O-Helix, which packs on the incoming dNTP (thick bonds).

### DETAILED DESCRIPTION

The invention provides polymerases that are useful in sequencing-by-synthesis reactions. Polymerases of the invention include mutations that make them more efficient at incorporating a nucleotide into a nascent strand of DNA or cDNA composed of at least one modified nucleotide compared to the wild-type polymerase. Other polymerases of the invention lack exonuclease activity or have reduced binding affinity of the 3′-5′ exonuclease domain.

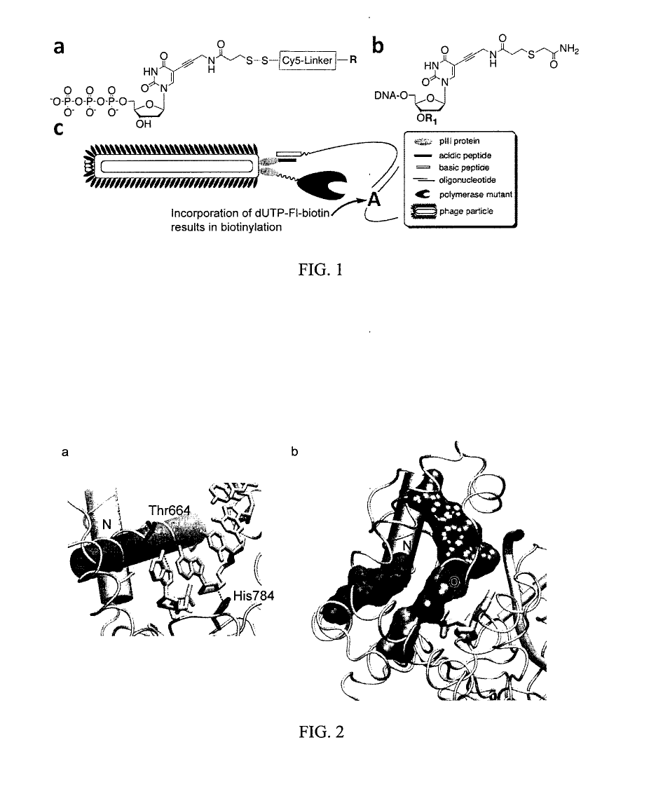

Nucleotide substrates for modified polymerases of the invention may be modified in numerous ways. For example, they may have modified 3′ hydroxyl groups or modified 2′ hydroxyl groups, they may have modified sugar portions, or preferably they may have modifications in the base portion of the nucleotide, such as an “overhang” of between 2 and 15 atoms (See, e.g., FIG. 1 panel B) that result from cleavage of a label or inhibitor upon prior incorporation. This “overhang” is also referred to as the scar, thus producing a scarred or scar-bearing nucleotide.

In certain embodiments, the modified nucleotide includes a detectable label connected to a nitrogenous base portion of a nucleotide by a cleavable linker, such as that shown in FIG. 1, panel A. FIG. 1 panel B provides an exemplary structure of the nucleotide shown in FIG. 1 panel A after cleavage of the label. The modified nucleotide shown in FIG. 1 panel B is an exemplary structure of a scarred or scar-bearing nucleotide. The chemical entity connected to the nitrogenous base portion of the nucleotide is the remaining portion of the linker after cleavage of the label, i.e., the scar.

Polymerases of the invention were identified using an activity-based selection system, that is described in the Examples below and is also depicted in FIG. 1 panel C. Such an activity-based selection system is also described in Fa et al. (J. Am. Chem. Soc. 126(6):1748-1754, 2004), Xia et al., (Proc. Natl. Acad. Sci. USA 99:6597-6602, 2002), and Leconte et al. (J. Am. Chem. Soc. 127:12470-12471, 2005). The contents of each of the references is incorporated by reference herein in their entirety.

Briefly, this system utilizes co-display of libraries of polymerase mutants and their oligonucleotide substrate on M13 phage. Phage production is optimized such that each phage particle displays zero to one polymerase mutant via fusion to a phagemid-encoded pIII, and four to five acidic peptides via fusions to the phage genome-encoded pIII. The displayed acidic peptides are used to attach oligonucleotide primers to the surface of the phage particle via a covalently-linked basic peptide. Because the pIII proteins are localized to one end of the phage particle, a displayed polymerase preferentially extends the primers that are covalently attached to the same phage particle, and when this occurs with a natural or modified dNTP that is biotinylated, it allows for selective recovery of the active polymerases and their respective genes using streptavidin beads. Thus, the selection system was used to enrich libraries in mutants possessing a desired activity, such as the recognition of detectably labeled nucleotides.

The activity-based selection system is utilized to enrich any library of mutated polymerases for possession of a desired activity, such as enhanced ability to incorporated detectably labeled nucleotides into a nascent strand of DNA or cDNA composed of at least one scar-bearing nucleotide. Exemplary polymerases include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9.degree.Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al, 1998, Proc. Natl. Acad. Sci. USA 95:14250).

Both mesophilic polymerases and thermophilic polymerases are contemplated. Thermophilic DNA polymerases include, but are not limited to, ThermoSequenase®, 9.degree.Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

In a particular embodiment, the polymerase is a Stoffel fragment of a Taq polymerase including at least one mutation that makes the Stoffel fragment (Sf) more efficient at incorporating a nucleotide into a nascent strand of DNA having at least one scar-bearing nucleotide compared to a native form of Taq polymerase. For example, the polymerase includes the following mutations in the Stoffel fragment: H784Q and T664A. In other embodiments, the polymerase includes the following mutations in the Stoffel fragment: F598I, I614F, V618I, L619M, 1638V, T640A, A643G, M646V, A661T, T664V, I665V, L670M, A691V, F700Y, I753V, T756S, A757G, H784Q. In other embodiments, the polymerase may include the following mutations in the Stoffel fragment: V618I, L619M, V631A, I638V, T640K, M646V, M658L, A661I, T664A, I665V, L670M, F700Y, A757G, H784Q. In other embodiments, the polymerase may include the following mutations in the Stoffel fragment: I614F, L619M, L622F, T640E, M678K, T684A, M751T, V753I, T756S, A757G, L760I.

The invention also provides mutated full length Taq polymerases, in which the mutations in the full length Taq polymerases provide enhanced ability to the mutated Taq polymerases, making these mutated polymerases more efficient at incorporating a nucleotide into a nascent strand of DNA or cDNA composed of at least modified nucleotide, e.g., a scar-bearing nucleotide, compared to a wild-type form of Taq polymerase. Enhanced ability may include at least about a 1-fold enhancement of incorporation efficiency as compared to the wild-type form of Taq, at least about a 5-fold enhancement, at least about a 10-fold enhancement, at least about a 40-fold enhancement, at least about a 50 fold enhancement, at least about a 100 fold enhancement, at least about a 300 fold enhancement, at least about a 400 fold enhancement, etc. In a particular embodiment, the enhancement ranges from about a 48-fold enhancement to about a 377-fold enhancement.

In certain embodiments, the mutations are present in a Stoffel fragment portion of the full length Taq polymerases. For example, the polymerase includes the following mutations in the Stoffel fragment: H784Q and T664A. In other embodiments, the polymerase includes the following mutations in the Stoffel fragment: F598I, I614F, V618I, L619M, I638V, T640A, A643G, M646V, A661T, T664V, I665V, L670M, A691V, F700Y, I753V, T756S, A757G, H784Q. In other embodiments, the polymerase may include the following mutations in the Stoffel fragment: V618I, L619M, V631A, I638V, T640K, M646V, M658L, A661I, T664A, I665V, L670M, F700Y, A757G, H784Q. In other embodiments, the polymerase may include the following mutations in the Stoffel fragment: I614F, L619M, L622F, T640E, M678K, T684A, M751T, V753I, T756S, A757G, L760I.

Because the polymerases of the invention are not adversely affected by scar-bearing nucleotides, the rate or extent of a sequencing reaction is not diminished after many rounds of polymerase mediate addition of detectably labeled nucleotides to a nascent strand of DNA or RNA. Thus, polymerases of the invention are useful for any sequencing technique, e.g., ensemble sequencing (Sanger sequencing) or single molecule sequencing by synthesis.

In other embodiments, polymerases of the invention lack or have reduced exonuclease activity or have reduced or eliminated binding affinity of the 3′-5′ exonuclease domain. DNA polymerase structure-function relationships have been extensively studied for over 20 years. Generally, DNA polymerases have three enzymatic activities in common: i) 5′-3′ exonuclease, ii) 3′-5′ exonuclease, and iii) 5′-3′ polymerase activity. Not all native polymerases have both of the exonuclease activities. The 5′-3′ exo activity degrades any nucleic acid strands base paired with the template as the enzyme processes along synthesizing a new complementary strand. The 3′-5′ exo activity is an editing function to remove bases which have been erroneously added (misincorporated) by the polymerase. The 5′-3′ pol activity is the enzyme activity which synthesizes the new strand of complementary nucleic acid copying the template and utilizing the 4 dNTPs (dATP, dCTP, dGTP and dTTP) to extend the primer from the 3′-end hydroxyl. These enzyme activities generally require cofactors, e.g. divalent metal ions such as magnesium, in order to function. It is widely known that within the polymerase structure there are discrete domains for each of the above mentioned enzyme activities.

The exonuclease activities are generally eliminated either by removing many amino acids from the gene sequence so as to remove an entire domain, e.g., by removing the 5′-3′exonuclease activity of E. coli Pol I (holoenzyme) conversion into Klenow fragment or Taq DNA pol conversion into Stoffel fragment, or selectively mutating one or more amino acids in the active site so as to drastically lower residual enzyme activity (<10⁻³-10⁻⁵ relative to normal), e.g., conversion of Klenow 3′-5′ exo+ into Klenow 3′-5′ exo− via mutation of the critical acidic amino acid(s) required for binding of the metal ion in the 3′-5′ exonuclease binding site.

The fidelity of template-dependent nucleic acid synthesis depends in part on the ability of the polymerase to discriminate between complementary and noncomplementary nucleotides, e.g., H-bonding between bases A:T and G:C. Normally, the conformation of the polymerase enzyme favors incorporation of the complementary nucleotide. However, there is still an identifiable rate of misincorporation even with polymerases that maintain minimally the 3′-5′ exonuclease editing activity that depends upon factors such as local sequence and the base to be incorporated. When the 3′-5′ exo activity in recombinants is diminished by mutations, misincorporation becomes potentially a bigger issue. In many cases when a misincorporation results, the polymerase activity is no longer able to effectively extend that strand resulting in a termination event. Termination can be somewhat controlled by varying the cofactors used when performing in vitro nucleic acid synthesis reactions, such as replacing the magnesium ion with manganese ion. When using magnesium as cofactor, a misincorporation is more likely to result in a termination and with manganese as the cofactor, the strand is more likely to continue extension following a misincorporation.

The ability of a polymerase to discriminate and edit the misincorporation involves the physical movement of the primer:template complex from the binding domain for polymerase activity to the binding domain of the 3′-5′ exo activity. Even in recombinant polymerases which have been mutated to eliminate the catalytic activity for the 3′-5′ exo, the 3′-5′ exo domain still retains the ability to bind nucleic acid (primer:template complex). This is not the same with the 5′-3′ exo domain mutants (Klenow or Stoffel), since the entire nucleic acid binding and catalytic domains no longer exists in these recombinants.

Synthetic or modified nucleotides and analogs, such as fluorescent dye labeled nucleotides, tend to be incorporated into a primer less efficiently than naturally-occurring dNTPs, due to the exonuclease activity of the polymerase. The reduced efficiency with which the unconventional nucleotides are incorporated by the polymerase adversely affects the performance of sequencing techniques that depend upon faithful incorporation of such unconventional nucleotides, ultimately leading to sequencing termination. Methods of the invention solve the problem of terminations in nucleic acid synthesis reactions by providing polymerases that lack exonuclease activity or have reduced binding affinity of the 3′-5′ exonuclease domain. Thus the yield of nucleic acid synthesis reactions can be improved by selectively mutating the polymerase enzyme so as to have reduced nucleic acid binding affinity in the 3′-5′ exo active site.

In certain embodiments the polymerase is Klenow fragment of E. coli Pol 1 or Stoffel fragment of Taq polymerase. In other embodiments, the polymerase includes at least one mutation that results in the lack of exonuclease activity or the reduced binding affinity of the 3′-5′ exonuclease domain. For example, the polymerase includes at least one mutation that results in absence of the 5′-3′ exonuclease activity of the polymerase. In other embodiments, the polymerase includes at least one mutation that results in absence of the 3′-5′ exonuclease activity of the polymerase.

In other embodiments, the polymerase comprises at least one mutation that results in reduced nucleic acid binding affinity of the 3′-5′ exonuclease domain of the polymerase. An example of such a polymerase is a klenow fragment including the following mutations: Leu361, Glu419, Lys422, Tyr423, Arg455, Phe473, and His660. See Lam et al. (Biochemistry 41:3943-3951, 2002). In certain embodiments, one or more of those amino acids is substituted with an Ala. Further information for structure:function variation of the 3′-5′ exonuclease activity may be found in Derbyshire et al. Meth Enz (1995) 262: 363-385. For those skilled in the art, there are in silico tools which align polymerase sequences from various sources and determine amino acids which perform similar structure:function relationships. Using these tools it is possible to transport mutations from one polymerase to another and obtain a similar outcome.

In other embodiments, the polymerase includes at least one mutation that affects metal ion binding. An example of a such a polymerase is a Klenow fragment including the following mutations: Asp355, Glu357, Asp424, and Asp501.

The polymerases described herein can be used in all sequencing reactions employing sequencing-by-synthesis approaches but is particularly useful in sequencing methods utilizing single molecule, sequencing-by-synthesis.

In a particular embodiment, the polymerases of the invention are used in a single-molecule sequencing-by-synthesis reaction. Single-molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Briefly, a single-stranded nucleic acid (e.g., DNA or cDNA) is hybridized to oligonucleotides attached to a surface of a flow cell. The oligonucleotides may be covalently attached to the surface or various attachments other than covalent linking as known to those of ordinary skill in the art may be employed. Moreover, the attachment may be indirect, e.g., via the polymerases of the invention directly or indirectly attached to the surface. The surface may be planar or otherwise, and/or may be porous or non-porous, or any other type of surface known to those of ordinary skill to be suitable for attachment. The nucleic acid is then sequenced by imaging the polymerase-mediated addition of fluorescently-labeled nucleotides incorporated into the growing strand surface oligonucleotide, at single molecule resolution. The following sections discuss general considerations for nucleic acid sequencing, for example, template considerations, polymerases useful in sequencing-by-synthesis, choice of surfaces, reaction conditions, signal detection and analysis.

### Nucleic Acid Templates

Nucleic acid templates include deoxyribonucleic acid (DNA) and/or ribonucleic acid (RNA). Nucleic acid templates can be synthetic or derived from naturally occurring sources. In one embodiment, nucleic acid template molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples for use in the present invention include viral particles or preparations. Nucleic acid template molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application Publication Number US2002/0190663 A1, published Oct. 9, 2003. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), .beta.-mercaptoethanol, DTE, GSH, cysteine, cysteamine, tricarboxyethyl phosphine (TCEP), or salts of sulfurous acid.

### Nucleotides

Nucleotides useful in the invention include any nucleotide or nucleotide analog, whether naturally-occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O—methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, and includes chain-terminating analogs. A nucleotide corresponds to a specific nucleotide species if they share base-complementarity with respect to at least one base.

Nucleotides for nucleic acid sequencing according to the invention preferably include a detectable label that is directly or indirectly detectable. Preferred labels include optically-detectable labels, such as fluorescent labels. Examples of fluorescent labels include, but are not limited to, 4-acetamido-4′-isothiocyanatostilbene-2,2′ disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Labels other than fluorescent labels are contemplated by the invention, including other optically-detectable labels.

### Nucleic Acid Polymerases

Any of the nucleic acid polymerases described herein may be mutated and identified using methods and techniques described herein to provide enhanced ability to incorporate a nucleotide into a nascent strand of DNA having at least one scar-bearing nucleotide compared to a native form of the polymerase. Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9.degree.Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1I/DP2 DNA polymerase II (Cann et al, 1998, Proc. Natl. Acad. Sci. USA 95:14250).

Both mesophilic polymerases and thermophilic polymerases are contemplated. Thermophilic DNA polymerases include, but are not limited to, ThermoSequenase®, 9.degree.Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit. Rev Biochem. 3:289-347 (1975)).

### Surfaces

In a preferred embodiment, nucleic acid template molecules are attached to a substrate (also referred to herein as a surface) and subjected to analysis by single-molecule sequencing as described herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Substrates are preferably coated to allow optimum optical processing and nucleic acid attachment. Substrates for use in the invention can also be treated to reduce background. Exemplary coatings include epoxides, and derivatized epoxides (e.g., with a binding molecule, such as an oligonucleotide or streptavidin).

Various methods can be used to anchor or immobilize the nucleic acid molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11:107-115, 1986. A preferred attachment is direct amine bonding of a terminal nucleotide of the template or the 5′ end of the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

### Detection

Any detection method can be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophor identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for imaging. TIRF microscopy uses totally internally reflected excitation light and is well known in the art. See, e.g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

### Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors. Any of a variety of other alignment techniques known to those of skill in the relevant art may also be used.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

### Materials and Methods

### Library Construction

Residues from an alignment of the sequences of six DNA Polymerase I analogs from: Thermus aquaticus, Thermus thermophilis, Thermus caldophilus, Thermus filiformis, Spirochaeta thermophila, and Thermomicrobium rosem that were identified to be within 14 Å of F667, and possessed natural variation were chosen for the 14A library. Details of the 21 residues chosen are provided in Tables 1 and 2 below. In Table 1, residues 640, 658, 818, 820, and 831 are located within 14 Å but were excluded from the library. These residues were, using Swiss PDB Viewer, identified to be >25% surface area exposed. It is expected that surface exposed residues may vary without selective advantage or disadvantage since they contribute little to the overall fold of the protein. These residues were manually observed in the crystal structure. Since none of the examined residues were located within close proximity to the active site or substrates, the highly surface exposed residues were excluded from the library. Residues 617, 682, and 706 are located within 14 Å but were excluded from the library as they are difficult to encode with degenerate codons.

TABLE 1

| Position | 618 | 619 | 622 | 631 | 636 | 638 | 641 | 643 |
|---|---|---|---|---|---|---|---|---|
| Amino acids in alignment | VLI | LM | FL | VA | EKR | IV | EQA | GA |
| Position | 646 | 657 | 661 | 663 | 664 | 668 | 669 | 700 |
| Amino acids in alignment | MVL | EKL | AI | TV | IV | IV | IML | FY |
| Position | 749 | 751 | 753 | 756 | 757 | 760 | | |
| Amino acids in alignment | VF | MT | ILV | TS | GA | LI | | |

TABLE 2

| Position | 618 | 619 | 622 | 631 | 638 | 640 | 643 | 646 | 658 | 661 | 663 |
|---|---|---|---|---|---|---|---|---|---|---|---|
| Amino acids | VLI | LM | FL | VA | IV | TAR | GA | MVL | QM | AI | TV |
| in nature | | | | | | | | | | | |
| Additional | | | | | | G | | | KL | VT | AI |
| amino acids | | | | | | | | | | | |
| Degenerate | VTA | MUG | YUC | GYA | RTT | RSG | GST | VTG | MWG | RYT | RYT |
| codon | | | | | | | | | | | |

| Position | 664 | 668 | 669 | 700 | 749 | 751 | 753 | 756 | 757 | 760 |
|---|---|---|---|---|---|---|---|---|---|---|
| Amino acids | IV | IV | IML | FY | VF | MT | ILV | TS | GA | LI |
| in nature | | | | | | | | | | |
| Additional | | | | | | | | | | |
| amino acids | | | | | | | | | | |
| Degenerate | RTT | RTT | MTK | TWT | KTT | AYG | VTA | WCU | GST | MUT |
| codon | | | | | | | | | | |

These 21 amino acids can be encoded into an approximately 600 base pair fragment, encoded by ten, 60 by primers in each direction. The primers were optimized to homogenize the predicted melting temperatures of overlapping regions as much as possible; in the final library, all overlapping regions annealed with a T_{M} of 67.5±5.5° C. (as calculated by the IDT Oligo Analyzer http://www.idtdna.com/analyzer/Applications/OligoAnalyzer/).

The primers were also designed to have the following criteria: no low usage codons; as little 3′ degeneracy as possible to avoid hybridization issues; and no SfiI or NotI restriction sites introduced. Primers used are listed in Table 3 below.

TABLE 3

F01 5′-CCACGGGCAGGCTAAGTAGCTCCGATCCCAACCTCCAGAACATCCCCRYTCRCACCCCGC-3′
F02 5′-TTGGGCAGAGGATCCGCCGGGCCTTCATCGCCGAGGAGGGGTGGCTATTGGTGGCCCTGG-3′
F03 5′-ACTACAGCCAGATCGAGCTCAGGVTTMTGGCCCACYTCTCCGGCGACGAGAACCTGATCC-3′
F04 5′-GGGYATTCCAGGAGGGGCGGGACRTTCACRMGGAGACCGSTAGCTGGVTGTTCGGCGTCC-3′
F05 5′-CCCGGGAAGCTGTCGATCCACGGMWGCGCCGCRYCGACAAGRYCRTCAACTTCGGGRTCM-3′
F06 5′-TKTACGGCATGTCGGCCCACCGCCTCTCCCAGGAGCTAGCCATCCCTTACGAGGAGGCCC-3′
F07 5′-AGGCCTTCATTGAGCGCTACTTTCAGAGCTWTCCCAAGGTGCGGGCCTGGATTGAGAAGA-3′
F08 5′-CCCTGGAGGAGGGCAGGAGGCGGGGGTACGTGGAGACCCTCTTCGGCCGCCGCCGCTACG-3′
F09 5′-TGCCAGACCTAGAGGCCCGGGTGAAGAGCGTGCGGGAGGCGGCCGAGCGTATGGCTKTTA-3′
F10 5′-ACAYGCCCVTTCAGGGCWCTGSTGCCGACMTTATGAAGCTGGCTATGGTGAAGCTCTTCC-3′
R01 5′-CGATGAAGGCCCGGCGGATCCTCTGCCCAAGCGGGGTGYGARYGGGGATGTTCTGGAGGT-3′
R02 5′-CCAKAABCCTGAGCTCGATCTGGCTGTAGTCCAGGGCCACCAATAGCCACCCCTCCTCGG-3′
R03 5′-YGTGAAYGTCCCGCCCCTCCTGGAATRCCCGGATCAGGTTCTCGTCGCCGGAGARGTGGG-3′
R04 5′-GGCGCWKCCGTGGATCGACAGCTTCCCGGGGGACGCCGAACABCCAGCTASCGGTCTCCK-3′
R05 5′-GGGAGAGGCGGTGGGCCGACATGCCGTAMAKGAYCCCGAAGTTGAYGRYCTTGTCGRYGC-3′
R06 5′-AGCTCTGAAAGTAGCGCTCAATGAAGGCCTGGGCCTCCTCGTAAGGGATGGCTAGCTCCT-3′
R07 5′-CGTACCCCCGCCTCCTGCCCTCCTCCAGGGTCTTCTGAATCCAGGCCCGCACCTTGGGAW-3′
R08 5′-CGCTCTTCACCCGGGCCTCTAGGTCTGGCACGTAGCGGCGGCGGCCGAAGAGGGTCTCCA-3′
R09 5′-KGTCGGCASCAGWGCCCTGAABGGGCRTGTTAAMAGCCATACGCTCGGCCGCCTCCCGCA-3′
R10 5′-GCATCCTGGCCCCCATTTCCTCCAGCCTGGGGAAGAGCTTCACCATAGCCAGCTTCATAA-3′

The 600 by fragment containing the library was obtained using standard protocols (Zhao et al., Nat. Protocols 1:1865-1871, 2006) built into a 1.8 kb fragment using additional assembly PCR reactions, and cloned into the pFAB vector.

Phage Preparation

X30 was derived from M13 helper phage by fusing of gIII to DNA encoding an acidic peptide that is used to attach substrate. All phage were at 30° C. to minimize the toxicity of expressed Sf protein. Wild-type Sf displaying phage was prepared using standard protocols (Xia et al., Proc. Natl. Acad. Sci. USA 99:6597-6602, 2002). To produce Sf library phage, 300 μL of SS320 competent cells (Sidhu et al., Methods Enzymol. 328:333-363, 2000) was electroporated with 10 μg of Sf library phagemid. The transformed cells were diluted in 250 mL of 2×YT, containing 7.5 μg/mL tetracycline, and 50 spectinomycin, and grown at 37° C. until the OD600 reached 0.6. The culture was inoculated with 75 μL of 10¹³ colony-forming units/mL X30 helper phage (M13 phage modified with a pIII-acidic peptide fusion, Xia et al., (Proc. Natl. Acad. Sci. USA 99:6597-6602, 2002) and Leconte et al. (J. Am. Chem. Soc. 127:12470-12471, 2005)) and incubated at 37° C. for 1 h without shaking. Following infection, the cells were centrifuged and resuspended in 250 mL fresh 2×YT with 50 μg/mL spectinomycin, 50 μg/mL kanamycin, and 0.4 mM isopropyl-D-thiogalactoside. The resuspended culture was grown at 30° C. overnight (approximately 16 h). The cells were centrifuged and the supernatant collected; the phage particles were isolated with two polyethylene glycol precipitations.

Synthesis of Basic Peptide-P50 DNA Conjugate

The basic peptide, K(GGS)₄AQLKKKLQALKKKNAQLKWKLQALKKKLAQGGC, whose affinity for the phage-displayed acidic peptide allows for coupling of the unnatural DNA substrate to the phage particle, contains an FMOC protected lysine (bold) and a photoprotected cysteine (underlined). The peptide was synthesized with Boc-protected amino acids (Calbiochem-Novabiochem), and, following deprotection of FMOC, coupled to maleimidocaproic acid (2-nitro 4-sulfo) phenyl ester (Bachem) on resin. Following HF cleavage, the crude product was purified by reverse-phase HPLC and characterized by ESI-MS. The 5′ thiol-modified oligonucleotide (5′-S-TTATG TATGT ATTTT CGACG TTTGC TAACA AGATA CGACT CACTA TAGGG) was synthesized by using a 5′ thiol reagent (S=5′-Thiol-Modifier C6, Glen Research, Sterling, Va.) and purified on a 12% denaturing polyacrylamide gel. Immediately prior to reaction, the oligonucleotide, referred to as P50, was deprotected according to the manufacturer's protocol. To conjugate P50 to the basic peptide, 300 μg of basic peptide dissolved in 150 μL of water was mixed with 75 μL of 1 M sodium phosphate (pH 7.0), 30 μL of 5 M NaCl, and 22 μL of 3 mM P50 oligonucleotide. The mixture was incubated at 50° C. for 16 h. The procedure was performed anaerobically to avoid oxidation. The peptide-P50 DNA conjugate was purified by ion exchange (Mono Q column, Bio-Rad).

DNA Substrate Attachment to Phage

In a typical preparation, 0.5 nmol peptide-P50 DNA conjugate was mixed with 20 μL, of 10×TBS and 2.5 nmol T28 template (5′-CCTCC AAGG (A)CCCTA TAGTG AGTCG TAT-NH₂ (purchased from IDT) and diluted to 200 μL with water. The mixture was heated rapidly to 85° C., cooled slowly to anneal primer P50 and template T28, and deprotected under 365-nm UV light (3 cm from a 4-W light source) for 45 min. The deprotected peptide-DNA conjugate was further diluted with 100 μL of attachment buffer (TBS containing 2.5 mM KCl, 1 mM EDTA, and 1 mM cystamine). To attach DNA substrate to phage, 300 μL of deprotected peptide-DNA conjugate was mixed with 200 μL of 5×10¹¹ colony-forming units/mL phage and incubated for 1 h at 37° C. The substrate-attached phage was polyethylene glycol-precipitated once to remove the free peptide-DNA conjugate.

Phage Selection

Following precipitation, the phage were resuspended in 50 μL 0.5×TBS. 35 μL of this solution (approximately 1×10¹¹ colony-forming units) was added to a final reaction volume of 200 μL, containing 50 mM Tris.HCl (pH 8.5), 50 mM KCl, 6.5 mM MgCl₂, and 1 μM virtual terminator dUTP. The reaction was initiated by transferring the mixture from ice to a 50° C. water bath. After 20 min of incubation, 100 μL of 0.5 M EDTA was added to stop the reaction, and the phage particles were polyethylene glycol-precipitated twice to remove unreacted biotin-UTP and subsequently resuspended in 200 μL of TBS. After removing residual precipitate by centrifugation (20,800×g for 10 min), the supernatant was added to 200 μL of streptavidin-coated magnetic beads (Dynal, Oslo). The beads were then washed 10 times with TBS containing 0.5% Tween-20. To release the bound phage, the beads were resuspended in 200 μL of TBS containing 10 mM MgCl₂ and 1 mg/mL DNase I and incubated at 37° C. for 1 hr. The phage present in the supernatant were titered and subjected to further amplification.

Mutant Polymerase Expression and Characterization (Small Scale)

After four rounds of selection, Sf inserts from recovered phagemids were cloned into a modified pET23b(+) vector (Novagen) for overexpression of Sf in E. coli BL21(DE3)pLysS (Promega). Single colonies were used to inoculate 2 mL of LB containing 100 μg/mL ampicilin and 34 μg/mL chloramphenicol. The inoculated culture was grown at 37° C. until the OD₆₀₀ reached 0.4-0.6. The cells were pelleted and resuspended in 2 mL of fresh LB supplemented with 100 μg/mL ampicilin, 34 μg/mL chloramphenicol, and 0.4 mM isopropyl-D-thiogalactoside. After an additional 4 h of growth, the induced cells were pelleted, lysed by resuspension in 200 μL Bugbuster (Novagen), incubated in a 75° C. water bath for 10 min, and centrifuged to remove cellular proteins. The partially purified Sf mutants were analyzed by SDS PAGE, and concentrations roughly adjusted for expression levels before being subjected to primer extension assays to screen for activity.

### General Polymerase Screen

Primer was 5′ radiolabeled with [γ³³P]-ATP (GE Biosciences) and T4 polynucleotide kinase (New England Biolabs). Primer-template duplexes were annealed in the reaction buffer by heating to 90° C. and slow cooling to room temperature. Assay conditions include: 40 nM template-primer duplex, 0.1-100 μM dNTP, 50 mM Tris buffer (pH 8.5), 50 mM KCl, 6.5 mM MgCl₂, and 50 μg/mL BSA. The reactions were initiated by adding the enzyme to the DNA/buffer/dNTP, incubated at 50° C. for 1-10 min, and quenched with 20 μL of loading buffer (95% formamide, 20 mM EDTA). The reaction mixture, (8 μL) was then analyzed by 15% polyacrylamide gel electrophoresis. Radioactivity was quantified using a Phosphorimager (Molecular Dynamics) with overnight exposures and the ImageQuant program. Percent conversion, used to quantify activity, is defined as the ratio of the density of the (n+1) band to the sum of the (n) and (n+1) bands.

Mutant Polymerase Expression and Purification (Large Scale)

Single colonies of Sf or Taq mutants were grown to saturation overnight in a 3 mL LB culture, supplemented with 100 μg/mL ampicilin and 34 μg/mL chlorophenicol, and then diluted in 300 mL of LB containing 100 μg/mL ampicilin and 34 μg/mL chlorophenicol and grown at 37° C. until the OD600 reached 0.4-0.6. Isopropyl-D-thiogalactoside was added to a final concentration of 0.4 mM to induce expression. After four hours, the overexpressed Sf or Taq proteins were partially purified by heat treatment and further purified with nickel chromatography. Protein eluted from the resin was buffer-exchanged into 50 mM Tris.HCl (pH 8.5), 0.5 mM EDTA, and concentrated to 0.5 mg/mL by using Centricon concentrators with a 30 kD or 50 kD membrane (Millipore) for Sf and Taq mutants, respectively. Protein concentrations were determined by Bradford assay (Bio-Rad).

General Polymerase Steady-State Kinetic Assay

Primer was 5′ radiolabeled with [γ³³P]-ATP (Amersham Biosciences) and T4 polynucleotide kinase (New England Biolabs). Primer-template duplexes were annealed in the reaction buffer by heating to 90° C. and slow cooling to room temperature. Assay conditions include: 40 nM template-primer duplex, 0.11-1.2 nM enzyme, 50 mM Tris buffer (pH 8.5), 50 mM KCl, 6.5 mM MgCl₂, and 50 μg/mL BSA. The reactions were initiated by adding the DNA-enzyme mixture to an equal volume (5 μl) of a 2×dNTP stock solution, incubated at 50° C. for 2-12 min, and quenched with 20 μL of loading buffer (95% formamide, 20 mM EDTA). The reaction mixture, (8 μL) was then analyzed by 15% polyacrylamide gel electrophoresis. Radioactivity was quantified using a Phosphorimager (Molecular Dynamics) with overnight exposures and the ImageQuant program. The kinetic data were fit to the Michaelis-Menten equation using the program Kaleidagraph (Synergy software). The data presented are averages of triplicates.

Determination of Polymerase Saturation

Polymerase saturation was determined by titrating polymerase concentration against 10 nM primer template DNA. Presteady state kinetic analysis of the incorporation of 100 nM dCTP was performed for each of the polymerase concentrations tested. The resultant rates were plotted against polymerase concentration. Nonlinear regression analysis was used to determine the maximum rate for the reaction. For subsequent experiments the saturating polymerase concentration was 1.2× the concentration at which the maximum rate was achieved.

Scarred Primer Preparation

Scarred primers were prepared enzymatically; following annealing of natural primers (bearing Rhodamine 110 dye-label, purchased from IDT) to templates, 15 U Klenow fragment (exo−) (New England Biolabs) in its supplied buffer was incubated with 5 μM DNA and 25 μM scarred dNTPs in 100 μL for 20 minutes at room temperature to incorporate scarred nucleotides onto the primer. Upon completion, reaction mixtures were heated at 70° C. for 15 minutes to inactivate Klenow fragment and purified from the scarred dNTPs using a DTR spin column (Edge BioSystems). Scarred primers were observed on an ABI3730 DNA sequencer and conversion to the desired scarred primer was greater than 95%.

General ‘Sequencing-Like’ Kinetic Assay

Polymerases were added to a mixture containing scarred DNA (prepared in previous section), modified dNTPs, and reaction buffer and incubated at either 37° C. or 50° C. and aliquots removed at variable time points and added directly to a quenching solution containing 100 mM EDTA. Reaction products were characterized on an ABI3730 sequencer using the GeneScan™ LIZ 120® size standard (Applied Biosystems) as a reference. The ratio of peak heights, obtained using ABI Peak Scanner (v1.0), was used to calculate percent primer conversion as a function of time and the data was fit to a first order exponential (Prism 4 by GraphPad).

### Example 2

### Activity Based Selection System for Selecting Polymerases of the Invention

Libraries of the N-terminal truncation of T. aquaticus DNA polymerase I (Stoffel fragment, Sf) were generated via synthetic shuffling (Ness et al., Nat. Biotechnol. 20:1251-1255, 2002 and Castle et al., Science 304(5674):1151-1154, 2004), of six homologous polymerases: Thermus aquaticus; Thermus thermophilis (91% amino acid identity to T. aquaticus); Thermus caldophilus (86%); Thermus filiformis (81%); Spirochaeta thermophila (54%); and Thermomicrobium rosem (54%). Mutations were restricted to twenty-one residues within 14 Å of an incoming dNTP (based on the ternary structure of T. aquaticus DNA polymerase I (Taq); Li et al., Proc. Natl. Acad. Sci. U.S.A. 96:9491-9496, 1999). The final library was constructed by assembly PCR and included 10⁸ chimeric Sf variants, with the quality of the library confirmed by sequencing random members.

To define selection conditions, the steady-state rates at which Sf extends a natural primer by incorporation of each dNTP-F1 (dNTP—fluorescently labeled) were characterized against its cognate base in the template. Table 3 below provides data that show the steady-state rates of incorporation of dNTP-F1s by Sf wt against a complementary nucleotide. In all cases, dNTP is complementary to dX.

TABLE 3
5′-dTAATACGACTCACTATAGGGAGA
3′-dATTATGCTGAGTGATATCCCTCT(X)GCTAGGTTACGGCAGGATCG
C

| dNTP-F1 | k_{cat} (min⁻¹) | K_{M} (μM) | k_{cat}/K_{M} (min⁻¹ M⁻¹) |
|---|---|---|---|
| A | 2.0 ± 0.2 | 0.79 ± 0.09 | 2.5 × 10⁶ |
| C | 1.9 ± 0.4 | 2.4 ± 0.7 | 7.8 × 10⁵ |
| G | 6.7 ± 1.2 | 0.33 ± 0.11 | 2.0 × 10⁷ |
| U | 1.3 ± 0.3 | 3.4 ± 1.3 | 3.8 × 10⁵ |

As an example, the incorporation of dUTP-F1 opposite dA in the template was examined, and biotinylated dUTP-F1 (FIG. 1 panel A) was synthesized and used for selections. Approximately 1×10¹¹ phage bearing both a polymerase mutant and a primer-template duplex containing a dA at the first templating position were prepared as described in Fa et al. (J. Am. Chem. Soc. 126(6):1748-1754, 2004), Xia et al., (Proc. Natl. Acad. Sci. USA 99:6597-6602, 2002), and Leconte et al. (J. Am. Chem. Soc. 127:12470-12471, 2005). Four rounds of selection were performed in which phage immobilization required the more efficient extension of the primer with the biotinylated dUTP-F1.

Based on a preliminary screen of 300 members of the enriched library, six mutants were prepared on a larger scale, and the rate of dUTP-F1 incorporation was measured using steady state kinetics (as shown in Table 4 below) as well as under sequencing-like conditions using a scarred primer. Table 4 provides data that show the steady-state rate of dUTP-F1 incorporation by Sf wt and Sf mutants.

TABLE 4
5′-dTAATACGACTCACTATAGGGAGA
3′-dATTATGCTGAGTGATATCCCTCT(A)GCTAGGTTACGGCAGGATCG
C

| | | | | |
|---|---|---|---|---|
| | | | | relative |
| Polymerase | k_{cat} (min⁻¹) | K_{M} (μM⁻¹) | k_{cat}/K_{M} (min⁻¹ μM⁻¹) | k_{cat}/K_{M}^{a} |

| | | | | |
|---|---|---|---|---|
| Sf wt | 1.7 ± 0.4 | 5.2 ± 1.0 | 3.3 × 10⁵ | 1.0 |
| Sf 281 | 6 ± 1.7 | 15.5 ± 5.2 | 3.9 × 10⁵ | 1.2 |
| Sf 292 | 12 ± 1 | 11.4 ± 1.8 | 1.1 × 10⁶ | 3.2 |
| Sf 247 | 12 ± 2 | 10.8 ± 0.9 | 1.1 × 10⁶ | 34 |
| Sf 197 | 15 ± 1 | 5.6 ± 2.3 | 2.7 × 10⁶ | 8.2 |
| Sf 267 | 15 ± 2 | 3.4 ± 1.3 | 4.4 × 10⁶ | 13.5 |
| Sf 168 | 21 ± 4 | 1.3 ± 0.4 | 1.6 × 10⁷ | 49.4 |

### Example 3

### Identification of Active Polymerases

The three most active polymerase mutants, Sf168, Sf197, and Sf267, showed an approximately 10- to 50-fold increased efficiency for dUTP-F1 incorporation and were characterized further with the other three modified dNTPs. Table 5 below shows mutations introduced into the Stoffel fragment for the three most active polymerase mutants, Sf168, Sf197, and Sf267. Mutations shown are Taq numbering.

TABLE 5

| Sf168 | Sf197 | Sf267 |
|---|---|---|
| F598I, I614F, V618I, L619M, | V618I, L619M, V631A, | I614F, L619M, L622F, |
| I638V, T640A, A643G, | I638V, T640K, M646V, | T640E, M678K, T684A, |
| M646V, A661T, T664V, | M658L, A661I, T664A, | M751T, V753I, T756S, |
| I665V, L670M, A691V, | I665V, L670M, F700Y, | A757G, and L760I |
| F700Y, I753V, T756S, | A757G, and H784Q | |
| A757G, and H784Q | | |

Table 6 below provides data that show rates of incorporation of dNTP-F1s by Sf wild type (WT) and Sf168, Sf197, and Sf267 and rates relative to that of wild type (WT) Sf polymerase. General reaction conditions are provided in Example 1. Buffer conditions were as follows: 20 mM Tris pH 8.8, 10 mM NaCl, 10 mM KCl, 10 mM ammonium sulfate, 0.1% Triton X-100, 10 mM MgCl₂, 50° C., 10 nM DNA, 100 nM dNTP-F1. Enzyme concentrations were chosen based on polymerase saturation concentration and were as follows: [Sfwt]=300 nM; [Sf167]=100 nM; [Sf197]=200 nM; [Sf267]=500 nM. Sequences of variable region in oligonucleotides were as follows: when dNTP=dATP-F1, N₃=dU, N₁=scar-dU, N₂=dA; when dNTP=dCTP-F1, N₃=dG, N₁=scar-dA, N=dU; when dNTP=dGTP-F1, N₃=dC, N₁=scar-dU, N₂=dA; when dNTP=dUTP-F1, N₃=dA, N₁=scar-dA, N₂=dU.

TABLE 6
5′-dTCCACTTATCCTTGCATCCATCCTCTGCCCTGN₁N₁N₁
3′-dAGGTGAATAGGAACGTAGGTAGGAGACGGGACN₂N₂N₂N₃TACTAT
CATTTGTACTATCATTTGTACTATCA

| | | | |
|---|---|---|---|
| WT | Sf168 | Sf197 | Sf267 |

| | | | | | | | |
|---|---|---|---|---|---|---|---|
| dNTP- | kₚₒₗ | kₚₒₗ | Relative | kₚₒₗ | Relative | kₚₒₗ | Relative |
| Fl1 (min⁻¹) | (min⁻¹) | to WT | (min⁻¹) | to WT | (min⁻¹) | to WT | |

| | | | | | | | |
|---|---|---|---|---|---|---|---|
| A | 0.14 | 10.51 | 78 | 4.94 | 37 | 1.59 | 12 |
| C | 0.18 | 4.66 | 26 | 4.25 | 24 | 2.91 | 16 |
| G | 1.05 | 8.19 | 8 | 9.11 | 9 | 7.22 | 7 |
| U | 0.04 | 2.32 | 55 | 0.76 | 18 | 0.54 | 13 |

These mutants incorporated the modified dNTPs 7- to 80-fold more efficiently than wild type Sf, showing that the selection did not introduce a bias for dUTP-F1 incorporation.

### Example 4

### Efficiency of Mutated Full Length Taq Polymerases

The mutations found in Sf168, Sf197, and Sf267 were cloned into full length Taq, and extension of a scarred primer terminus was characterized by measuring the rate of incorporation of each dNTP-F1 by Taq or a selected mutant. Taq197 (corresponding to the mutations from Sf197) was the most optimized of the three polymerase mutants, incorporating each dNTP-F1 48- to 377-fold more efficiently than wild type Taq, with no apparent bias toward the identity of the dNTP-F1 or the sequence of the primer, as shown in Table 7 below. Buffer conditions were as follows: 20 mM Tris pH 8.8, 10 mM NaCl, 10 mM KCl, 10 mM ammonium sulfate, 0.1% Triton X-100, 10 mM MgCl₂, 37° C., 40 nM enzyme, 10 nM DNA, 100 nM dNTP-F1. N₁ nucleotides are scarred (see FIG. 1 panel B).

TABLE 7
5′TCCACTTATCCTTGCATCCATCCTCTGCCCTGN₁N₁N₁
3′-AGGTGAATAGGAACGTAGGTAGGAGACGGGACN₂N₂N₂N₃TACTATC
ATTTGTACTATCATTTGTACTATCA

| | | | | |
|---|---|---|---|---|
| | | Taq | Taq197 | Relative rate |
| dNTP-F1 | (N₁N₁N₁/N₂N₂N₂)^{b} | (min⁻¹) | (min⁻¹) | (Taq197/Taq) |

| | | | | |
|---|---|---|---|---|
| N = A | UUU/AAA | 0.011 ± 0.002 | 1.4 ± 0.2 | 133 |
| C | AAA/UUU | 0.002 ± 0.0002 | 0.66 ± 0.12 | 377 |
| G | UIJU/AAA | 0.14 ± 0.05 | 6.6 ± 1.8 | 48 |
| U | CCC/GGG | 0.05 ± 0.02 | 2.9 ± 1.2 | 58 |

### Example 5

### Fidelity of Taq197

To examine the fidelity of Taq197, the misincorporation of the three incorrect dNTP-F1s opposite dA in the template was characterized. Importantly, as with wild type Taq, Taq197 does not measurably synthesize any of the mispairs, even after 90 minutes. Thus, based on the detection limit of the assay, it was possible to set an upper limit of 5.6×10⁻⁴ min⁻¹ for the rate of mispair formation, making correct incorporation of modified nucleotides more than 5,000-fold more efficient than mispair formation. These data show that the fidelity of Taq197 has not been compromised and that it is more than sufficient for sequencing applications.

### Example 6

### Characterization of Taq197

Taq197 has 14 mutations relative to its wild type progenitor (See FIG. 2 and Table 5 above). Based on the crystal structure of the ternary complex of the wild type enzyme and natural substrates, at least two of the mutations, H784Q and T664A, alter direct interactions with the modified substrates. The side chain of His784 hydrogen-bonds with the ribosyl oxygen of the primer terminus (FIG. 2 panel A); mutation to glutamine preserves this hydrogen-bond while simultaneously repositioning the primer terminus to better accommodate the scar. Thr664 is located in the developing major groove of the DNA, 6.1 Å away from the site where the linker is attached to the incoming dNTP (FIG. 2 panel A); mutation to the smaller alanine allows for the polymerase to better tolerate the bulky linker.

In addition to these direct interactions, there are a number of more subtle changes; five amino acids in the O-helix and three in the N-helix, which packs on the O-helix, are mutated to other hydrophobic residues (FIG. 2 panel B). These mutations appear to participate in three clusters of packing interactions that contribute to improved positioning of the O-helix, which has been shown to close over, and make specific contacts with, the incoming dNTP during DNA synthesis (Li et al., Proc. Natl. Acad. Sci. U.S.A. 96:9491-9496, 1999; Li et al., G., EMBO J. 17:7514-7525, 1998; and Loh et al. DNA Repair (Amst.) 4:1390-1398, 2005).

Data herein show that the Taq197 polymerase has acquired an expanded substrate repertoire by optimizing both direct and indirect contacts with the modified substrates. Taq197 was selected from a library of Sf mutants for increased recognition of the next generation single molecule sequencing by synthesis reactions. Considering the significant increase in activity with the labeled nucleotides relative to that of the wild type Taqpolymerase, Taq197 has immediate value for single molecule sequencing.

In addition, Taq197 and any further optimized progeny will also facilitate a variety of different single molecule sequencing and other technologies in which polymerase recognition of similarly modified nucleotides is required (Turcatti et al., Nucleic Acids Res. 36:e25, 2008; and Mitra et al., Anal. Biochem. 320:55-65, 2003).

What is claimed is:

1. A polymerase enzyme comprising at least one mutation that enhances ability of the polymerase as compared to a wild-type polymerase to incorporate a nucleotide into a nascent strand of DNA or cDNA comprising at least one modified nucleotide.

2. The polymerase according to claim 1, wherein the polymerase is a DNA polymerase.

3. The polymerase according to claim 1, wherein the modified nucleotide comprises a modification in the base portion of the nucleotide.

4. The polymerase according to claim 3, wherein the modified nucleotide comprises a residue of a cleavable linker attaching a detectable labeled to a nitrogenous base portion of the nucleotide.

5. The polymerase according to claim 4, wherein the detectable label is a fluorescent label.

6. The polymerase according to claim 5, wherein the fluorescent label is selected from the group consisting of cyanine, rhodamine, fluorescien, coumarin, BODIPY, alexa, and conjugated multi-dyes.

7. The polymerase according to claim 1, wherein the polymerase is a Taq polymerase comprising mutations that enhance ability of the polymerase to incorporate a nucleotide into a nascent strand of DNA or cDNA comprising at least one modified nucleotide compared to a wild-type Taq polymerase.

8. The polymerase according to claim 7, wherein the polymerase comprises the following mutations in the Taq polymerase: H784Q and T664A.

9. The polymerase according to claim 7, wherein the polymerase comprises the following mutations in the Taq polymerase: F598I, I614F, V618I, L619M, I638V, T640A, A643G, M646V, A661T, T664V, I665V, L670M, A691V, F700Y, I753V, T756S, A757G, H784Q.

10. The polymerase according to claim 7, wherein the polymerase comprises the following mutations in the Taq polymerase: V618I, L619M, V631A, I638V, T640K, M646V, M658L, A661I, T664A, I665V, L670M, F700Y, A757G, H784Q.

11. The polymerase according to claim 7, wherein the polymerase comprises the following mutations in the Taq polymerase: I614F, L619M, L622F, T640E, M678K, T684A, M751T, V753I, T756S, A757G, L760I.

12. A Taq polymerase comprising the following mutations: V618I, L619M, V631A, I638V, T640K, M646V, M658L, A661I, T664A, I665V, L670M, F700Y, A757G, H784Q.

13. A Taq polymerase comprising the following mutations: I614F, L619M, L622F, T640E, M678K, T684A, M751T, V753I, T756S, A757G, L760I.

14. A Taq polymerase comprising the following mutations: V618I, L619M, V631A, I638V, T640K, M646V, M658L, A661I, T664A, I665V, L670M, F700Y, A757G, H784Q.

15. A method of sequencing a nucleic acid, the method comprising:
directly or indirectly anchoring a nucleic acid duplex to a surface, the duplex comprising a primer portion and a template portion having at least one modified nucleotide at its 3′ terminus;
exposing the duplex to at least one detectably labeled nucleotide in the presence of a modified polymerase capable of catalyzing addition of the nucleotide to the duplex;
detecting incorporation of the nucleotide into the primer portion; and
repeating the exposing and detecting steps at least once.

16. The method according to claim 15, further comprising: determining a sequence of the template based upon the order of incorporation of the labeled nucleotides.

17. The method according to claim 15, further comprising: removing unincorporated nucleotide and polymerase in all or some repetitions of the exposing and detecting steps.

18. The method according to claim 15, further comprising: neutralizing the label on the labeled nucleotide after the detecting step.

19. The method according to claim 15, wherein the template is individually optically resolvable.

20. The method according to claim 15, wherein the template is attached to the surface.

21. The method according to claim 15, wherein the nucleotide is a non-native nucleotide.

22. A method for increasing the accuracy of sequencing a nucleic acid, the method comprising:
contacting a nucleic acid duplex comprising a primer nucleic acid hybridized to a template nucleic acid with a polymerase enzyme in the presence of a first labeled nucleotide under conditions that permit the polymerase to add nucleotides to said primer in a template-dependent manner, wherein the polymerase has reduced or eliminated exonuclease activity or has reduced or eliminated binding affinity of the 3′-5′ exonuclease domain;
detecting a signal from the incorporated labeled nucleotide; and
sequentially repeating said contacting and detecting steps at least once, wherein sequential detection of incorporated labeled nucleotide determines the sequence of the nucleic acid.

23. The method according to claim 22, wherein said duplex is attached to a surface.

24. The method according to claim 23, wherein said surface comprises a plurality of duplex immobilized at different positions on the substrate.

25. The method according to claim 24, wherein at least some of said duplex are individually optically resolvable.

26. The method according to claim 22, wherein the label is a fluorescent label.

27. The method according to claim 22, wherein the polymerase is Klenow fragment of E. coli Pol 1 or Stoffel fragment of Taq polymerase.

28. The method according to claim 22, wherein the polymerase comprises at least one mutation that results in the lack of exonuclease activity or the reduced binding affinity of the 3′-5′ exonuclease domain.

29. The method according to claim 22, wherein the polymerase comprises at least one mutation that results in absence of the 5′-3′ exonuclease activity of the polymerase.

30. The method according to claim 22, wherein the polymerase comprises at least one mutation that results in absence of the 3′-5′ exonuclease activity of the polymerase.

31. The method according to claim 22, wherein the polymerase comprises at least one mutation that results in reduced nucleic acid binding affinity of the 3′-5′ exonuclease domain of the polymerase.

32. The method according to claim 31, wherein the polymerase is a klenow fragment comprising the following mutations: Leu361, Glu419, Lys422, Tyr423, Arg455, Phe473, and His660.

33. The method according to claim 32, wherein Ala is substituted at any of the positions.

34. The method according to claim 22, wherein the polymerase comprises at least one mutation that affects metal ion binding.

35. The method according to claim 34, wherein the polymerase is a Klenow fragment comprising the following mutations: Asp355, Glu357, Asp424, and Asp501.


SEQUENCE LISTING (31 sequences)

SEQ ID NO: 1 - DNA, 66 bp, Artificial Sequence
  primer sequence
ccacgggcaggctaagtagctccgatcccaacctccagaacatccccnnnnnntcnnncaccccgc

SEQ ID NO: 2 - DNA, 60 bp, Artificial Sequence
  primer sequence
ttgggcagaggatccgccgggccttcatcgccgaggaggggtggctattggtggccctgg

SEQ ID NO: 3 - DNA, 66 bp, Artificial Sequence
  primer sequence
actacagccagatcgagctcaggnnnttnnntggcccacnnntctccggcgacgagaacctgatcc

SEQ ID NO: 4 - DNA, 72 bp, Artificial Sequence
  primer sequence
gggnnnattccaggaggggcgggacnnnttcacnnnnnnggagaccgnnntagctggnnntgttcggcgtcc

SEQ ID NO: 5 - DNA, 78 bp, Artificial Sequence
  primer sequence
cccgggaagctgtcgatccacggnnnnnngcgccgcnnnnnncgacaagnnnnnncnnntcaacttcgggnnntcnnn

SEQ ID NO: 6 - DNA, 62 bp, Artificial Sequence
  primer sequence
tnnntacggcatgtcggcccaccgcctctcccaggagctagccatcccttacgaggaggccc

SEQ ID NO: 7 - DNA, 62 bp, Artificial Sequence
  primer sequence
aggccttcattgagcgctactttcagagctnnntcccaaggtgcgggcctggattgagaaga

SEQ ID NO: 8 - DNA, 60 bp, Artificial Sequence
  primer sequence
ccctggaggagggcaggaggcgggggtacgtggagaccctcttcggccgccgccgctacg

SEQ ID NO: 9 - DNA, 62 bp, Artificial Sequence
  primer sequence
tgccagacctagaggcccgggtgaagagcgtgcgggaggcggccgagcgtatggctnnntta

SEQ ID NO: 10 - DNA, 70 bp, Artificial Sequence
  primer sequence
acannngcccnnnttcagggcnnnctgnnntgccgacnnnttatgaagctggctatggtgaagctcttcc

SEQ ID NO: 11 - DNA, 66 bp, Artificial Sequence
  primer sequence
cgatgaaggcccggcggatcctctgcccaagcggggtgnnngannnnnnggggatgttctggaggt

SEQ ID NO: 12 - DNA, 64 bp, Artificial Sequence
  primer sequence
ccannnaannncctgagctcgatctggctgtagtccagggccaccaatagccacccctcctcgg

SEQ ID NO: 13 - DNA, 68 bp, Artificial Sequence
  primer sequence
nnngtgaannngtcccgcccctcctggaatnnncccggatcaggttctcgtcgccggagannngtggg

SEQ ID NO: 14 - DNA, 70 bp, Artificial Sequence
  primer sequence
ggcgcnnnnnnccgtggatcgacagcttcccgggggacgccgaacannnccagctannncggtctccnnn

SEQ ID NO: 15 - DNA, 76 bp, Artificial Sequence
  primer sequence
gggagaggcggtgggccgacatgccgtannnannngannncccgaagttgannngnnnnnncttgtcgnnnnnngc

SEQ ID NO: 16 - DNA, 60 bp, Artificial Sequence
  primer sequence
agctctgaaagtagcgctcaatgaaggcctgggcctcctcgtaagggatggctagctcct

SEQ ID NO: 17 - DNA, 62 bp, Artificial Sequence
  primer sequence
cgtacccccgcctcctgccctcctccagggtcttctcaatccaggcccgcaccttgggannn

SEQ ID NO: 18 - DNA, 60 bp, Artificial Sequence
  primer sequence
cgctcttcacccgggcctctaggtctggcacgtagcggcggcggccgaagagggtctcca

SEQ ID NO: 19 - DNA, 72 bp, Artificial Sequence
  primer sequence
nnngtcggcannncagnnngccctgaannngggcnnntgttaannnagccatacgctcggccgcctcccgca

SEQ ID NO: 20 - DNA, 60 bp, Artificial Sequence
  primer sequence
gcatcctggcccccatttcctccagcctggggaagagcttcaccatagccagcttcataa

SEQ ID NO: 21 - protein, 46 aa, Artificial Sequence
  Basic peptide-P50 DNA conjugate
KGGSGGSGGSGGSAQLKKKLQALKKKNAQLKWKLQALKKKLAQGGC

SEQ ID NO: 22 - DNA, 50 bp, Artificial Sequence
  Synthetic oligonucleotide
ttatgtatgtattttcgacgtttgctaacaagatacgactcactataggg

SEQ ID NO: 23 - DNA, 28 bp, Artificial Sequence
  T28 template
cctccaaggaccctatagtgagtcgtat

SEQ ID NO: 24 - DNA, 23 bp, Artificial Sequence
  primer sequence
taatacgactcactatagggaga

SEQ ID NO: 25 - DNA, 45 bp, Artificial Sequence
  Template sequence
attatgctgagtgatatccctctngctaggttacggcaggatcgc

SEQ ID NO: 26 - DNA, 23 bp, Artificial Sequence
  primer sequence
taatacgactcactatagggaga

SEQ ID NO: 27 - DNA, 45 bp, Artificial Sequence
  Template sequence
attatgctgagtgatatccctctagctaggttacggcaggatcgc

SEQ ID NO: 28 - DNA, 35 bp, Artificial Sequence
  primer sequence
tccacttatccttgcatccatcctctgccctgnnn

SEQ ID NO: 29 - DNA, 68 bp, Artificial Sequence
  Template sequence
aggtgaataggaacgtaggtaggagacgggacnnnntactatcatttgtactatcatttgtactatca

SEQ ID NO: 30 - DNA, 35 bp, Artificial Sequence; identical to SEQ ID NO: 28
  primer sequence

SEQ ID NO: 31 - DNA, 68 bp, Artificial Sequence; identical to SEQ ID NO: 29
  Template sequence